(19) United States
(12) Patent Application Publication
(10) Pub. No.: US 2007/0123925 A1
(43) Pub. Date: May 31, 2007

(54) Pre-curved guiding catheter with mechanically actuated anchor

(75) Inventors: Thierry Benjamin, Lowell, MA (US); James Coyle, Castlegal (IE)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/287,879

(22) Filed: Nov. 28, 2005

(51) Int. Cl. A61M 29/00

(52) U.S. Cl. 606/194

(57) ABSTRACT

A guiding catheter includes an elongate shaft with a central bore and a mechanically expandable braided anchor mounted about the distal end of the shaft. The guiding catheter has a pre-formed curve adjacent the distal end of the shaft. A sheath disposed about the catheter is selectively slidable along the catheter shaft to expand the anchor into engagement with the wall of an artery. Methods of using the guiding catheter are also disclosed.

### FIELD OF THE INVENTION

The present invention relates generally to an intraluminal guiding catheter used in a medical procedure, and more particularly, to a guiding catheter with a mechanically-actuated distal anchor for enhancing back-up force provided by a pre-formed curve.

### BACKGROUND OF THE INVENTION

A stenosis, or narrowing of a blood vessel such as a coronary artery may comprise a hard, calcified substance and/or a softer thrombus material. There have been numerous therapeutic procedures developed for the treatment of stenosis in a coronary artery. One of the better-known procedures is percutaneous transluminal coronary angioplasty (PTCA). According to this procedure, the narrowing in the artery can be reduced by positioning a dilatation balloon across the stenosis and inflating the balloon to re-establish acceptable blood flow through the artery. Additional therapeutic procedures may include stent deployment, atherectomy, and thrombectomy, which are well known and have proven effective in the treatment of such stenotic lesions.

The therapeutic procedure starts with the introduction of a guiding catheter into the cardiovascular system from a convenient vascular access location, such as through the femoral artery in the groin area or other locations in the arm or neck. The guiding catheter is advanced through the arteries until its distal end is located near the stenosis that is targeted for treatment. During PTCA, for example, the distal end of the guiding catheter is typically inserted only into the ostium, or origin of the coronary artery. A guidewire is advanced through a central bore in the guiding catheter and positioned across the stenosis. An interventional therapy device, such as balloon dilatation catheter, is then slid over the guidewire until the dilatation balloon is properly positioned across the stenosis. The balloon is inflated to dilate the artery. To help prevent the artery from re-closing, a physician can implant a stent inside the artery. The stent is usually delivered to the artery in a compressed shape on a stent delivery catheter and expanded by a balloon to a larger diameter for implantation against the arterial wall.

At times it is difficult to advance the interventional catheter across the stenosis because the narrowing may be very tight or the vessel(s) may have significant bends to be negotiated between the ostium and the target stenosis. In such difficult cases, the guiding catheter can fail to provide sufficient structural support or “backup” as the interventional catheter is pushed distally against resistance. In failing to provide backup support, the guiding catheter reacts by deforming such that the catheter tip “backs out,” proximally from its initial position at the ostium of the branch artery. When the guiding catheter distal end remains in a fixed position, it facilitates the ability to advance the interventional catheter. As guiding catheters have advantageously evolved to have thinner walls and smaller outside diameters, it has been increasingly challenging to provide the necessary “backup support” in all clinical cases.

A catheter system that may be utilized to increase the backup support of a conventional guiding catheter is described in U.S. Pat. No. 5,484,412 to Pierpont. An anchoring catheter extends through and distally beyond a guiding catheter into a blood vessel targeted for interventional therapy. The anchoring catheter has two longitudinally-spaced external balloons. A first balloon may be inflated within the guiding catheter to temporarily secure the anchoring catheter within the guiding catheter. A second balloon may be inflated within the vessel to temporarily secure the anchoring catheter within the vessel. Simultaneous inflation of the first and second balloons anchors the guiding catheter position with respect to the vessel. An interventional catheter, such as an angioplasty catheter may be passed through a lumen of the anchoring catheter and into the vessel. The anchoring catheter occupies space in the guiding catheter lumen that could otherwise be used to reduce the outer diameter of the guiding catheter or permit use of a larger interventional device. An accessory apparatus is required to inflate and deflate the balloons on the anchoring catheter.

A guiding catheter having increased backup support is described in U.S. Pat. No. 4,832,028 to Patel. Typical of most guiding catheters, the Patel catheter is pre-curved at the distal end to set and hold a supporting position in the vasculature while the therapeutic catheter crosses and treats the lesion. Additionally, the Patel catheter includes a balloon disposed around the guiding catheter distal end that, when appropriately positioned, may be inflated to lock the catheter tip in place within the ostium of a coronary artery. The Patel catheter requires a balloon inflation lumen in addition to the main lumen. The inflation lumen occupies space in the guiding catheter that could otherwise be used to reduce the outer diameter of the guiding catheter or permit use of a larger interventional device. An accessory apparatus is required to inflate and deflate the locking balloon.

There is a need to selectively secure the distal end of a guiding catheter to its position in the ostium of a branch vessel, so that an interventional catheter can be housed therein and advanced distally into the branch vessel without losing structural support or backup from the guiding catheter. The guiding catheter should have an anchoring system that may be activated and deactivated simply and quickly without accessories during interventional catheterization procedures. It is desirable for the anchoring system to occupy minimal space in, or contribute as little as possible to the overall diameter of the guiding catheter. Other desirable features and characteristics of the present invention will become apparent from the subsequent detailed description and the appended claims taken in conjunction with the accompanying drawings.

### BRIEF SUMMARY OF THE INVENTION

The invention provides a guiding catheter with a mechanically-actuated braided anchor mounted at the distal end. The guiding catheter includes an elongate shaft having a central bore and a pre-curved region adjacent the distal end. A sheath is slidably disposed about the shaft. A braided anchor is attached between the distal ends of the shaft and the sheath. The braided anchor is responsive to longitudinal movement between the ends of the anchor to transform between a collapsed configuration and an expanded configuration, the expanded configuration having a centrally located major diameter sized for engaged apposition with a vessel wall of a patient.

A method is disclosed for using the inventive guiding catheter with mechanically actuated anchor. The method includes providing a guiding catheter having the embodiment described above; inserting the guiding catheter into the vascular system of the patient and positioning the anchor proximal to the stenotic lesion to be treated; and moving the sheath along the shaft to expand the anchor into engagement with the wall of the artery to provide temporary anchoring of the guiding catheter tip.

In other embodiments of the invention, the method may also include: inserting a therapeutic device through the central bore of the guiding catheter; positioning the therapeutic portion of the therapeutic device across the stenosis; and treating the stenosis with the therapeutic device.

### BRIEF DESCRIPTION OF THE DRAWINGS

The following drawings are illustrative of particular embodiments of the invention and therefore do not limit its scope. They are presented to assist in providing a proper understanding of the invention. The drawings are not to scale and are intended for use in conjunction with the explanations in the following detailed descriptions. Like reference numerals denote like elements in the drawings, wherein;

FIG.1 is a side view of a distal region of a guiding catheter in accordance with the invention, shown with an anchor in a collapsed configuration;

FIG. 2 is a side view of a distal region of the guiding catheter shown in FIG. 1, shown with the anchor in an expanded configuration

FIG. 3 is a transverse cross-sectional view of the guiding catheter shown in FIG. 2, taken along line 2-2;

FIG. 3 is a side view of a distal portion of the guiding catheter of FIG. 1, shown with the anchor in a contracted configuration;

FIG. 4 illustrates a guiding catheter in accordance with the invention, shown deployed in the cardiovascular system of a patient; and

FIGS. 5-7 illustrate the use of the inventive guiding catheter in a diseased vessel during a typical angioplasty procedure.

### DETAILED DESCRIPTION OF THE INVENTION

Specific embodiments of the present invention are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

The following detailed description is merely exemplary in nature and is not intended to limit the invention or the application and uses of the invention. Although the description of the invention is in the context of treatment of blood vessels such as the coronary, carotid and renal arteries, the invention may also be used in any other passageways where it is deemed useful to provide temporary anchoring of the catheter tip. Furthermore, there is no intention to be bound by any expressed or implied theory presented in the preceding technical field, background, brief summary or the following detailed description.

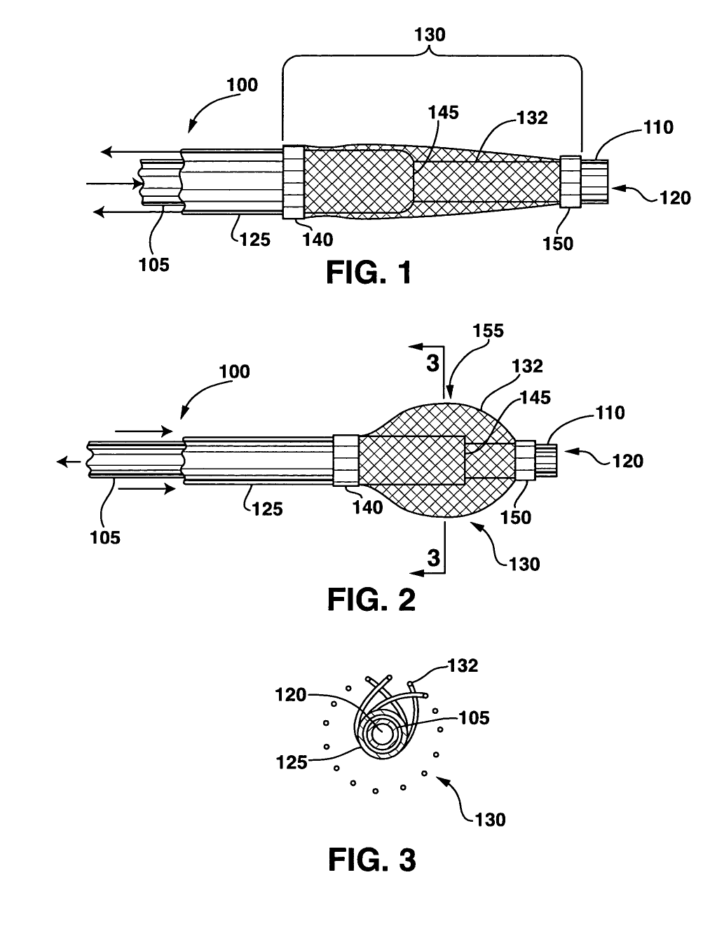

FIGS. 1 and 2 illustrate a distal region of one embodiment of guiding catheter 100, including elongate shaft 105 with distal end 110, which may comprise an optional soft tip. Bore 120 extends through shaft 105 between open proximal and distal ends, has a low-friction surface and is sized and shaped to receive and direct there through a variety of treatment devices such as guidewires and/or therapeutic devices including, but not limited to balloon catheters or stent delivery systems. Elongate sheath 125 is slidably disposed about shaft 105, terminating adjacent shaft distal end 110.

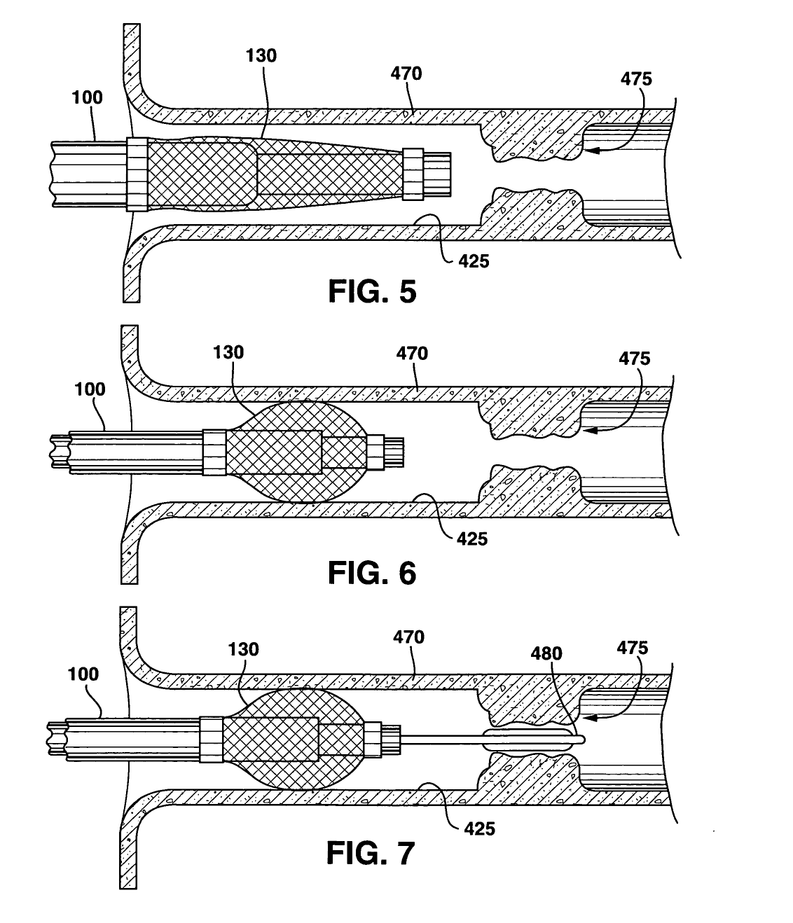

Anchor 130 is mounted about a distal region of catheter 100 and includes tubular braid 132. Braid proximal end 140 is affixed adjacent sheath distal end 145 and braid distal end 150 is affixed adjacent shaft distal end 110. In FIG. 1, tubular braid 132 is shown in a collapsed configuration. In FIG. 2, tubular braid 132 is shown in an expanded configuration having a broadest transverse section, or major diameter 155. Sliding sheath 125 proximally or distally along shaft 105 translates braid ends 140, 150 apart or together causing transformation of braid 132 between expanded and collapsed configurations. In the unrestricted expanded configuration, major diameter 155 is greater than the diameter of a branch vessel lumen being intubated, such that braid 132 engages the vessel wall, as will be described in further detail below in conjunction with FIGS. 5-7.

Catheter shaft 105 is a flexible tube that is designed to advance through a patient's vasculature to remote arterial locations without buckling or undesirable bending. As is well known to those of skill in the art, catheter shaft 105 includes a pre-formed distal curve that can provide enhanced “backup support” as therapeutic catheters are advanced through bore 120 of guiding catheter 100 and across a stenosis. Any one of a number of pre-formed curve shapes may be incorporated into guiding catheter 100, such as Judkins-type or Amplatz-type curves, as non-limiting examples. Curve 160 may be pre-formed utilizing various known methods including, but not limited to, the method disclosed in U.S. Pat. No. 5,902,287 entitled “Guiding Catheter and Method of Making Same.”

Catheter shaft 105 may be constructed of one or more flexible biocompatible materials, including, but not limited to, polyethylene, polypropylene, polyurethane, polyesters, or polyethylene block amide copolymer. Catheter shaft 105 may also include a layer of braided filaments that resist kinking and enhance longitudinal transmission of rotation. To further aid in advancing guiding catheter 100 through the patient's vasculature, it may be desirable to vary the stiffness of catheter shaft 105 by varying the braid pitch, by varying the properties of materials used in construction, or by combining both techniques.

Bore 120 of guiding catheter 100 may provide a slippery interior surface for reducing frictional forces between the interior surface and devices that may be moved through bore 120. In one exemplary embodiment, the interior surface is provided with a slippery coating, such as a silicone compound or a hydrophilic polymer. In another exemplary embodiment, the interior surface includes a liner formed from a slippery material. Those with skill in the art may appreciate that any one of numerous low-friction, biocompatible materials such as, for example, fluoropolymers (e.g. PTFE, FEP), polyolefins (e.g. polypropylene, high-density polyethylene), or polyamides, may be used for bore 120. 100251 Sheath 125 may comprise flexible, thin-walled biocompatible materials such as those mentioned above with respect to shaft 105. One example of a suitable material for sheath 125 is tubing having 0.051 mm (0.002 inch) wall thickness and extruded from 7233D PEBAX® resin from Arkema, Inc., Philadelphia, Pa., U.S.A. Further-more, to provide a small overall diameter of guiding catheter 100, sheath 125 may comprise very thin thermoset polyimide tubing, which has sufficient stiffness to provide precise manual actuation of anchor 130 by pushing or pulling sheath 125 relative to shaft 105, as indicated by the force vectors in FIGS. 1 and 2.

Tubular braid 132 may comprise braided filaments that may be made from a high-modulus thermoplastic or thermo-set plastic, nitinol (TiNi), stainless steel or a work-hardenable super alloy comprising nickel, cobalt, chromium and molybdenum. Braid proximal and distal ends 140, 150 may be fixed to sheath 125 and catheter shaft 105, respectively, by any suitable manner known in the art, such as epoxy or cyanoacrylate adhesives. Radiopaque material may be incorporated into one or both of the adhesive bonds, either as a solid marker band or as particulate filler material in the adhesive. Braid proximal end 140 may abut or be located directly around the distal end of sheath 125. Alternatively, proximal end 140 may be spaced somewhat proximally from the distal end of sheath 125, as illustrated in FIGS. 1 and 2, to provide an intermediate step in both diameter and stiffness along the assembly. Braid distal end 150 may also be inset into a groove (not shown) formed in the outer wall of catheter shaft 105, thus reducing stiffness and overall profile of the bond between braid distal end 150 and catheter shaft 105. The groove in catheter shaft 105 may be formed as taught in U.S. Pat. No. 6,059,769 to Lunn et al., which is incorporated by reference herein in its entirety. Tubular braid 132 allows unrestricted fluid flow through the interstices thereof.

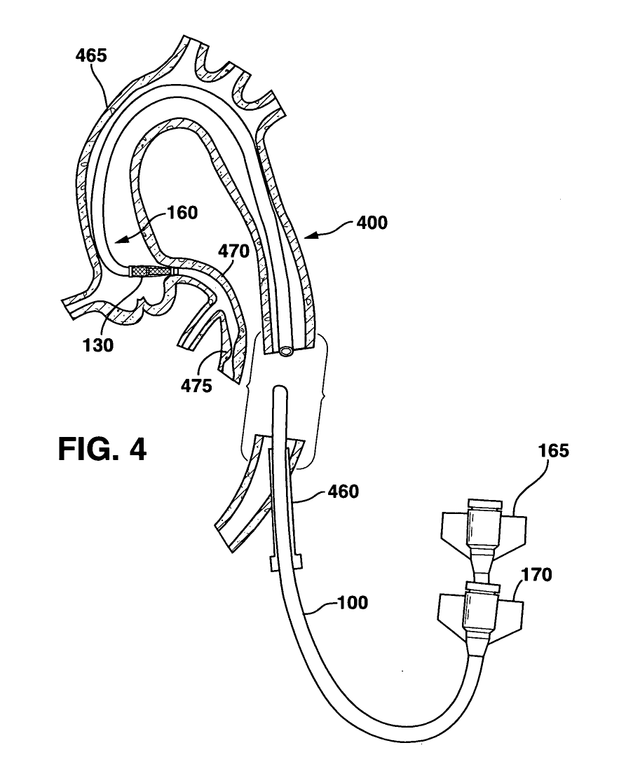

As shown in FIG. 4, connector fitting 165 is coupled to, and provides a functional access port at the proximal end of guiding catheter 100. Fitting 165 is attached to shaft 105 and has a central opening in communication with bore 120 to allow passage of therapeutic devices there through. Connector fitting 165 may be made of metal or of a hard polymer (e.g. medical grade polycarbonate, polyvinyl chloride, acrylic, acrylonitrile butadiene styrene (ABS), or polyamide) that possesses the requisite structural integrity, as is well known to those of skill in the art.

Control fitting 170 is coupled to the proximal end of sheath 125 and has a central opening to allow shaft 105 to slide there through. Fitting 170 provides an enlarged component for the clinician to manually grasp when sliding sheath 125 along shaft 105 to actuate anchor 130. Optionally, fitting 170 may include a mechanism (not shown) for temporarily locking shaft 105 and sheath 125 in their relative longitudinal positions that define either the expanded or collapsed configurations of anchor 130. For example, control fitting 170 can include a Touhy-Borst type fitting with adjustable gland for frictionally gripping shaft 105 at the desired position. Fitting 170 may be made of the same or similar material as those mentioned above with respect to connector fitting 165.

An exemplary method of using guiding catheter 100 will now be described. FIG. 4 illustrates guiding catheter 100 positioned within patient's vascular system 400 for use with a therapeutic device. The clinician confirms that anchor 130 is in the compressed configuration and inserts the distal end of guiding catheter 100 through introducer sheath 460 into vascular system 400, typically through a femoral artery in the groin area. Guiding catheter 100 is advanced through aorta 465 until the distal end of the catheter is located in the ostium of targeted branch artery 470. In the example shown, branch artery 470 is a patient's left coronary artery.

If the clinician elects to anchor guiding catheter 100 during the intervention, then the distal end of guiding catheter 100 is inserted into artery 470 until anchor 130 is substantially within artery 470, as illustrated in FIG. 5. At least the portion of anchor 130 that will become, when in the expanded configuration, major diameter 155 is positioned distal to the ostium. The clinician manually separates connector fitting 165 and control fitting 170 to actuate anchor 130, thus transforming it from the collapsed configuration to the expanded configuration, which provides locking, or temporary securement between anchor 130 and vessel wall 425, as illustrated in FIG. 6. Connector fitting 165 and control fitting 170 are manually separated according to the clinician's preference: Shaft 105 may be held stationary in artery 470 while sheath 125 is advanced, or sheath 125 may be held stationary in artery 470 while shaft 105 is withdrawn.

A therapeutic device, such as balloon dilatation catheter 480, including a dilatation balloon, is advanced through bore 120 until the balloon reaches a desired position within stenosis 475, as illustrated in FIG. 7. The dilatation balloon is then inflated to dilate stenosis 475. Balloon dilatation catheter 480 may then be deflated and removed. Lastly, guiding catheter 100 is withdrawn from vessel lumen 420.

While at least one exemplary embodiment has been presented in the foregoing detailed description, it should be understood that various changes can be made in the function and arrangement of elements without departing from the scope of the invention as set forth in the appended claims and the legal equivalents thereof.

What is claimed is:

1. A guiding catheter for intubation and selective anchoring in a branch vessel in a patient, the vessel having a lumen and a lumen wall, the catheter comprising:
an elongate hollow shaft having open proximal and distal ends and a pre-curved region adjacent the shaft distal end;
an elongate sheath slidably disposed about the shaft and having a sheath distal end terminating adjacent the shaft distal end; and
a braided anchor mounted about the shaft and having an anchor distal end fixed adjacent the shaft distal end and an anchor proximal end fixed adjacent the sheath distal end, wherein relative longitudinal movement between the ends of the anchor accompanies transformation of the anchor between a collapsed configuration and an expanded configuration sized for anchoring contact with the vessel wall.

2. The guiding catheter of claim 1, wherein the elongate hollow shaft has a reinforcement layer.

3. The guiding catheter of claim 2, wherein the reinforcement layer comprises a tubular braid.

4. The guiding catheter of claim 1, wherein the anchor is selectively transformable between the collapsed configuration and the expanded configuration by sliding the sheath longitudinally along the shaft.

5. The guiding catheter of claim 1 further comprising a connector fitting mounted at the shaft proximal end in communication with a bore extending between the open proximal and distal shaft ends.

6. A method of using a guiding catheter comprising:
providing a guiding catheter in accordance with claim 1;
inserting the catheter shaft distal end into a vascular system of the patient;
advancing the catheter shaft distal end to the branch vessel in the patient;
intubating the vessel with the catheter shaft distal end; and
sliding the sheath distally along the shaft to transform the anchor into the expanded configuration wherein the anchor contacts the vessel wall.

7. The method of claim 6 further comprising:
inserting a therapeutic device through the guiding catheter; and
operating the therapeutic device to treat the patient from within the branch vessel.

8. The method if claim 6, wherein the therapeutic device is an angioplasty catheter and operating the therapeutic device comprises inflating a balloon to dilate a stenosis in the vessel.

9. The method of claim 6 further comprising:
sliding the sheath proximally along the shaft to transform the anchor into the collapsed configuration; and
withdrawing the catheter shaft from the vascular system of the patient.
